(19) United States
(12) Patent Application Publication
(10) Pub. No.: US 2006/0286181 A1
(43) Pub. Date: Dec. 21, 2006

(54) Diet supplements for causing fast weight loss, improving daytime energy, promoting nighttime relaxation and sleep, controlling appetite and/or increasing metabolism

(76) Inventors: Paul T. Gardiner, Mississauga (CA); Marvin A. Heuer, Mississauga (CA)

Correspondence Address: KENYON & KENYON LLP, ONE BROADWAY, NEW YORK, NY, 10004, US

(21) Appl. No.: 11/448,535

(22) Filed: Jun. 5, 2006

Related U.S. Application Data
(60) Provisional application No. 60/691,661, filed on Jun. 17, 2005.

(51) Int. Cl. A61K 36/82; A61K 36/28; A61K 31/19; A61K 31/685; A61K 31/555; A61K 31/522

(52) U.S. Cl. 424/729; 424/777; 424/761; 424/764; 424/778; 514/263.31; 514/557; 514/78; 514/184

(57) ABSTRACT

A diet supplement is provided in two parts. One part is provided in a daytime formulation comprising at least the Calcium and Potassium double salt of Garcinia Cambogia Extract, and Green Tea Leaf Extract. The second part is provided in a night time formulation comprising at least the Calcium and Potassium double salt of Garcinia Cambogia Extract and Melatonin. The prevent invention provides a daily diet cycle for enhancing fast weight loss, improving day-time energy, promoting nighttime relaxation and sleep, controlling appetite and increasing metabolism.

### RELATED APPLICATIONS

The application is related to and claims benefit of priority to Applicant's co-pending U.S. Provisional Patent Application Ser. No. 60/691,661 entitled “Diet Supplement for Causing Rapid Weight Loss, Improving Day Time Energy, Promoting Nighttime Relaxation and Sleep, Controlling Appetite and/or Increasing Metabolism,” filed Jun. 17, 2005, the disclosure of which is hereby fully incorporated by reference.

### FIELD OF THE INVENTION

The present invention relates to a diet supplement, that includes the calcium and potassium double salt of Garcinia Cambogia and one of Green Tea Leaf Extract or Melatonin. In addition, the present invention relates to a method for causing fast weight loss, improving daytime energy, promoting nighttime relaxation and sleep, controlling appetite and/or increasing metabolism comprising the step of consuming the diet supplement. In addition, the present invention relates to a method of manufacturing the diet supplement.

### SUMMARY OF THE INVENTION

The present invention provides for a diet supplement for causing fast weight loss, improving daytime energy, promoting nighttime relaxation and sleep, controlling appetite and/or increasing metabolism. The diet supplement is particularly well suited for mothers, e.g., new mothers after childbirth, having a heightened need for fast weight loss, supplemental daytime energy, improved nighttime relaxation and sleep, more controlled appetite and/or an increased metabolism. Advantageously, the diet supplement provides a 24-hour benefit by way of two compositions. For example, the diet supplement may provide two or more different compositions, together comprising one daily diet supplement cycle. A first composition comprising at least the Calcium and Potassium double salt of Garcinia Cambogia Extract supplying 60% Hydroxycitric Acid, and Green Tea Leaf Extract may be a daytime formula and may provide benefits most needed by an individual during the day. A second composition comprising at least the Calcium and Potassium double salt of Garcinia Cambogia Extract supplying 60% Hydroxycitric Acid, and Melatonin may be provided as a nighttime formula and may provide benefits most needed by an individual during the night.

The present invention also provides, by the consumption of the diet supplement, a method for causing fast weight loss, improving daytime energy, promoting nighttime relaxation and sleep, controlling appetite and/or increasing metabolism.

In addition, the present invention relates to a method of manufacturing a diet supplement for causing fast weight loss, improving daytime energy, promoting nighttime relaxation and sleep, controlling appetite and/or increasing metabolism. In one embodiment, there is provided a method of manufacturing a first composition of a diet supplement, the first composition comprising at least the Calcium and Potassium double salt of Garcinia Cambogia Extract supplying 60% Hydroxycitric Acid, and Green Tea Leaf Extract. In a second embodiment, there is provided a method of manufacturing a second composition of a diet supplement, the second composition comprising at least the Calcium and Potassium double salt of Garcinia Cambogia Extract supplying 60% Hydroxycitric Acid, and Melatonin.

### DETAILED DESCRIPTION OF THE INVENTION

The present invention, according to various embodiments thereof, is directed to a diet supplement for inducing fast weight loss, improving daytime energy, promoting nighttime relaxation and sleep, controlling appetite and/or increasing metabolism.

The diet supplement is particularly well suited for mothers, e.g., new mothers after childbirth, having a heightened need for fast weight loss, supplemental daytime energy, improved nighttime relaxation and sleep, more-controlled appetite and/or an increased metabolism. Advantageously, the diet supplement provides a 24-hour benefit by way of two compositions. For example, the diet supplement may provide two or more different compositions which together comprise one daily diet supplement cycle. A first composition may be a daytime formula and may provide benefits most needed by an individual during the day. A second composition may be a nighttime formula and may provide benefits most needed by an individual during the night. More specifically, the diet supplement may include a first, e.g., daytime, composition to be consumed early in the daytime such as prior to breakfast and/or lunch, and a second, e.g., nighttime, composition to be consumed prior to the nighttime such as prior to dinner.

Calcium and Potassium Double Salt of Garcinia Cambogia (Supplying 60% HCA)

Hydroxycitric acid (HCA) is extracted from the fruit of the Garcinia Cambogia plant. It is used to control weight by virtue of its ability to inhibit fat production and suppress appetite.

U.S. Pat. No. 6,875,891, entitled “Process for Preparing Highly Water Soluble Double Salts of Hydroxycitric Acid Particularly Alkali and Alkaline Earth Metal Double Salts,” the disclosure of which is hereby fully incorporated by reference, describes a method for producing highly water-soluble Calcium and Potassium Hydroxycitric Acid salts which are odorless and essentially tasteless. The process involves the steps of precipitating sparingly soluble alkaline earth metal salts of Hydroxycitric acid from an aqueous extract of plants belonging to the Garcinia species, dissolving said alkaline earth metal salts in aqueous alkali and adjusting the pH of said alkaline solution by adding an extract of purified Garcinia fruit extract. From this process, the Calcium salt of Hydroxycitric Acid can be precipitated. Additionally, said Calcium salt can be treated with Potassium Hydroxide to form the Potassium and Calcium double salt of Hydroxycitric Acid which can further be purified by treatment with activated charcoal, filtered and spray dried.

HCA has been shown to inhibit fatty acid synthesis and repress appetite in rats (Watson, J. A., M. Fang, and J. M. Lowenstein, Tricarballylate and hydroxycitrate: substrate and inhibitor of ATP: citrate oxaloacetate lyase. Arch Biochem Biophys, 1969. 135(1): p. 209-17., Louter-van de Haar, J., et al., Comparison of the effects of three different (−)-hydroxycitric acid preparations on food intake in rats. Nutr Metab (Lond), 2005. 2: p. 23.). It is additionally known to be a competitive inhibitor of ATP:citrate lyase; an enzyme necessary for the conversion of carbohydrates into fat. By inhibiting this enzyme, HCA blocks the body's ability to produce fat from carbohydrate sources. Garcinia Cambogia extract has also been shown to improve glucose metabolism in mice (Hayamizu, K., et al., Effect of Garcinia cambogia extract on serum leptin and insulin in mice. Fitoterapia, 2003. 74(3): p. 267-73.).

Several human clinical trials have demonstrated safety and beneficial effects of HCA in terms of weight management. HCA clinical studies have shown that its administration is able to produce a reduction caloric intake (Westerterp-Plantenga, M. S. and E. M. Kovacs, The effect of (−)-hydroxycitrate on energy intake and satiety in overweight humans. Int J Obes Relat Metab Disord, 2002. 26(6): p. 870-2.) while further increasing fat oxidation during exercise in untrained men and women (Lim, K., et al., (−)-Hydroxycitric acid ingestion increases fat utilization during exercise in untrained women. J Nutr Sci Vitaminol (Tokyo), 2003. 49(3): p. 163-7., Tomita, K., et al., (−)-hydroxycitrate ingestion increases fat oxidation during moderate intensity exercise in untrained men. Biosci Biotechnol Biochem, 2003. 67(9): p. 1999-2001.). A randomized controlled trial which combined data from two earlier trials demonstrated that daily chromium supplementation with moderate exercise over a period of 8 weeks resulted in increased weight loss as compared to a placebo group and an improved blood cholesterol profile (Preuss, H. G., et al., Efficacy of a novel calcium/potassium salt of (−)-hydroxycitric acid in weight control. Int J Clin Pharmacol Res, 2005. 25(3): p. 133-44.). Additionally, levels of serotonin, a neurotransmitter which when low provides a hunger signal particularly associated with carbohydrate cravings, were significantly increased by HCA. This could result in a reduction of carbohydrate cravings.

In an embodiment of the present invention, which is set forth in greater detail in Example 1 below, the first, e.g., daytime, composition of the diet supplement may include the Calcium and Potassium double salt of Garcinia Cambogia Extract supplying 60% Hydroxycitric Acid. A serving of the first, e.g., daytime, composition of the diet supplement comprises from at least about 1 mg to about 10 g of the Calcium and Potassium double salt of Garcinia Cambogia Extract supplying 60% Hydroxycitric Acid. The preferred dosage of a serving of the first, e.g., daytime, composition of the diet supplement comprises about 1.555 g of the Calcium and Potassium double salt of Garcinia Cambogia Extract supplying 60% Hydroxycitric Acid.

In an embodiment of the present invention, which is set forth in greater detail in Example 2 below, the second, e.g., nighttime, composition of the diet supplement may include the Calcium and Potassium double salt of Garcinia Cambogia Extract supplying 60% Hydroxycitric Acid. A serving of the second, e.g., daytime, composition of the diet supplement may include from about 10 g of the Calcium and Potassium double salt of Garcinia Cambogia Extract supplying 60% Hydroxycitric Acid. In a preferred dosage, a serving of the second, e.g., nighttime, composition of the diet supplement comprises about 1.555 g of the Calcium and Potassium double salt of Garcinia Cambogia Extract supplying 60% Hydroxycitric Acid.

Green Tea (Catechins, ECGC, Norepinephrine Increase)

The active compounds of Green Tea are a family of polyphenols (Catechins) wherein tannins are the largest of the group. The most active specific compound is epigallocatechin gallate (ECGC) which makes up 10-50% of the total Catechins. Green tea also contains caffeine, although typically significantly less than black tea.

Green Tea mainly acts in a beneficial way through the polyphenol's antioxidant activities as evidenced by several laboratory studies. One clinical study has shown that ingestion of green tea extract results in a rapid increase in plasma antioxidant activity (Benzie, I. F., et al., Consumption of green tea causes rapid increase in plasma antioxidant power in humans. Nutr Cancer, 1999. 34(1): p. 83-7.). Moreover, Green Tea has also been shown to be effective in aiding weight loss (Chantre, P. and D. Lairon, Recent findings of green tea extract AR25 (Exolise) and its activity for the treatment of obesity. Phytomedicine, 2002. 9(1): p. 3-8.). This effect may be due to two activities. Firstly, Green tea both reduces fat digestion and secondly, it increases energy expenditure (Berube-Parent, S., et al., Effects of encapsulated green tea and Guarana extracts containing a mixture of epigallocatechin-3-gallate and caffeine on 24 h energy expenditure and fat oxidation in men. Br J Nutr, 2005. 94(3): p. 432-6.). The increase in energy expenditure may result from fat stores via the oxidation of fat (thermogenesis) (Choo, J. J., Green tea reduces body fat accretion caused by high-fat diet in rats through beta-adrenoceptor activation of thermogenesis in brown adipose tissue. J Nutr Biochem, 2003. 14(11): p. 671-6, Dulloo, A. G., et al., Efficacy of a green tea extract rich in catechin polyphenols and caffeine in increasing 24-h energy expenditure and fat oxidation in humans. Am J Clin Nutr, 1999. 70(6): p. 1040-5.). The thermogenic activity of Green Tea may be greatly enhanced by synergistic cooperation with caffeine (Dulloo, A. G., et al., Green tea and thermogenesis: interactions between catechin-polyphenols, caffeine and sympathetic activity. Int J Obes Relat Metab Disord, 2000. 24(2): p. 252-8.).

The mechanism of action of Green Tea may be, at least partially, due to an increase in norepinephrine. Catechins are known to inhibit catechol-O-methyl-transferase (COMT), an enzyme that degrades norepinephrine (Borchardt, R. T. and J. A. Huber, Catechol O-methyltransferase. 5. Structure-activity relationships for inhibition by flavonoids. J Med Chem, 1975. 18(1): p. 120-2.). In turn, norepinephrine inhibits degradation of intracellular cyclic AMP (cAMP), an important signaling molecule involved in many metabolic processes including thermogenesis.

The first, e.g., daytime, composition of the diet supplement includes Green Tea Dry Leaf Extract (camellia sinensis). A serving of the first, e.g., daytime, composition of the diet supplement may include about 1.0 mg to about 10 g of Green Tea Dry Leaf Extract (camellia sinensis). The preferred dosage of the first, e.g., daytime, composition of the diet supplement includes about 304.44 mg of Green Tea Dry Leaf Extract (camellia sinensis).

Anhydrous Caffeine

Caffeine is a naturally occurring xanthine alkaloid found in some plants where it serves as a natural pesticide. In humans, however, it may have numerous beneficial effects, the most common of which uses caffeine as a supplement to the central nervous system. In this capacity, it is used as a stimulant and performance enhancer. A meta-analysis complied from forty double-blind studies support the use of caffeine to increase physical endurance (Doherty, M. and P. M. Smith, Effects of caffeine ingestion on exercise testing: a meta-analysis. Int J Sport Nutr Exerc Metab, 2004. 14(6): p. 626-46.; Graham, T. E., E. Hibbert, and P. Sathasivam, Metabolic and exercise endurance effects of coffee and caffeine ingestion. J Appl Physiol, 1998. 85(3): p. 883-9.; Kovacs, E. M., J. Stegen, and F. Brouns, Effect of caffeinated drinks on substrate metabolism, caffeine excretion, and performance. J Appl Physiol, 1998. 85(2): p. 709-15.).

Caffeine is also widely used to control weight, which may occur through multiple mechanisms. Significant weight loss related to caffeine supplementation has been observed in obese women (Yoshida, T., et al., Relationship between basal metabolic rate, thermogenic response to caffeine, and body weight loss following combined low calorie and exercise treatment in obese women. Int J Obes Relat Metab Disord, 1994. 18(5): p. 345-50.) which may be, at least in part, due to increased lipolysis as fat is metabolized (Jung, R. T., et al., Caffeine: its effect on catecholamines and metabolism in lean and obese humans. Clin Sci (Lond), 1981. 60(5): p. 527-35.). Caffeine has additionally been shown to increase the basal metabolic rate (Roberts, A. T., et al., The effect of an herbal supplement containing black tea and caffeine on metabolic parameters in humans. Altern Med Rev, 2005. 10(4): p. 321-5.) wherein this also adds to its weight-lowering effects.

Biochemically, caffeine as it is structurally similar, binds to, but does not activate, adenosine receptors which are normally activated by adenosine to induce sleep (Shi, D., et al., Chronic caffeine alters the density of adenosine, adrenergic, cholinergic, GABA, and serotonin receptors and calcium channels in mouse brain. Cell Mol Neurobiol, 1993. 13(3): p. 247-61.).

The first, e.g., daytime, composition of the first, e.g., daytime, composition of the diet supplement may include anhydrous caffeine. A serving of the first, e.g., daytime, composition of the diet supplement may include from about 0.1 mg to about 1000 mg of anhydrous caffeine. In a preferred dosage, a serving of the first, e.g., daytime, composition of the diet supplement comprises about 75 mg of anhydrous caffeine.

Chromium Polynicotinate

Chromium is an essential trace mineral that is used to control blood sugar levels by aiding insulin, which can help control or reduce weight. Chromium, as it is poorly absorbed by the body, must therefore be combined with a more efficiently absorbed compound such as niacin (found in polynicotinate). Chromium likely exerts its main function as a component of the glucose tolerance factor, which is involved in insulin sensitivity.

Chromium has been shown clinically to additionally increase lean mass (Bahadori, B., et al., [Effect of chromium yeast and chromium picolinate on body composition of obese, non-diabetic patients during and after a formula diet]. Acta Med Austriaca, 1997. 24(5): p. 185-7.) and reduce body fat when combined with exercise (Grant, K. E., et al., Chromium and exercise training: effect on obese women. Med Sci Sports Exerc, 1997. 29(8): p. 992-8.). Furthermore, Chromium has also been shown to increase HDL (‘good’) cholesterol (Riales, R. and M. J. Albrink, Effect of chromium chloride supplementation on glucose tolerance and serum lipids including high-density lipoprotein of adult men. Am J Clin Nutr, 1981. 34(12): p. 2670-8.).

Moreover, in an animal study designed to assess the effectiveness of Chromium Polynicotinate supplementation on Metabolic Syndrome, as defined by a cluster of risk factors including obesity, increased abdominal fat, insulin resistance, hypertension and blood lipid abnormalities, it was shown that compared to control groups Chromium Polynicotinate was effective in lowering systolic blood pressure (Talpur N., et al., Effects of Niacin-bound chromium, maitake mushroom fraction SX and (−)-Hydroxycitric acid on the Metabolic Symdrome in Aged Zucker Fatty Rats. Mol and Cell Biochem, 2003. 252 (1-2): p. 369-77.). This study also observed less free radical damage in the liver and kidney tissue as compared to controls, indicating antioxidant properties. Additionally, this study suggests Chromium supplementation may also benefit persons suffering from diabetes. Further to the animal studies, a clinical study has shown that Niacin-bound Chromium supplementation for 8 weeks is able to result in a significant loss of body fat in overweight subjects as compared to a placebo group, while sparing muscle from loss (Crawford V., et al., Supplementation on body composition in overweight African-American women. Diabetes and Obes Metab, 1999. 1(6): p. 331-37.).

In addition or alternatively, the first, e.g., daytime, composition of the diet supplement may include Chromium Polynicotinate. A serving of the first, e.g., daytime, composition of the diet supplement may include from about 1 mcg to about 1000 mcg of Chromium Polynicotinate. In a preferred dosage, a serving of the first, e.g., daytime, composition of the diet supplement comprises about 133 mcg of Chromium Polynicotinate.

In addition or alternatively, the second, e.g., night-time, composition of the diet supplement may include Chromium Polynicotinate. A serving of the second, e.g., nighttime, composition of the diet supplement may include from about 1 mcg to about 1000 mcg of Chromium Polynicotinate. In a preferred dosage, a serving of the second, e.g., nighttime, composition of the diet supplement comprises about 133 mcg of Chromium Polynicotinate.

Gymnema Sylvestre

Gymnema Sylvestre is a plant used in traditional Eastern medicine to treat diabetes by virtue of its ability to inhibit glucose absorption and suppress the taste of ‘sweetness’. In mice, a peptide isolated from Gymnema Sylvestre has been shown to specifically inhibit the mice's response to sucrose (Ninomiya, Y. and T. Imoto, Gurmarin inhibition of sweet taste responses in mice. Am J Physiol, 1995. 268(4 Pt 2): p. R1019-25.). Moreover, Gymnema Sylvestre extracts have also been shown to suppress cellular glucose uptake in various animal models (Shimizu, K., et al., Suppression of glucose absorption by some fractions extracted from Gymnema sylvestre leaves. J Vet Med Sci, 1997. 59(4): p. 245-51.).

Gymnema Sylvestre extract in conjunction with insulin has been shown to be more effective than insulin alone at controlling blood glucose levels in patients with insulin-dependent diabetes mellitus, Shanmugasundaram, E. R., et al., Use of Gymnema sylvestre leaf extract in the control of blood glucose in insulin-dependent diabetes mellitus. J Ethnopharmacol, 1990. 30(3): p. 281-94.). It has further been shown to raise insulin levels in such patients (Baskaran, K., et al., Antidiabetic effect of a leaf extract from Gymnema sylvestre in non-insulin-dependent diabetes mellitus patients. J Ethnopharmacol, 1990. 30(3): p. 295-300.) with insulin-dependent diabetes mellitus. Gymnema Sylvestre extract has also been combined with HCA to produce greater weight loss (Preuss, H. G., et al., Effects of a natural extract of (−)-hydroxycitric acid (HCA-SX) and a combination of HCA-SX plus niacin-bound chromium and Gymnema sylvestre extract on weight loss. Diabetes Obes Metab, 2004. 6(3): p. 171-80.) in experimental settings.

The first, e.g., daytime, composition of the diet supplement may include Gymnema Sylvestre Leaf Extract. A serving of the first, e.g., daytime, composition of the diet supplement may include from about 1 mg to about 2000 mg of Gymnema Sylvestre Leaf Extract. In a preferred dosage, a serving of the first, e.g., daytime, composition of the diet supplement comprises about 133 mg of Gymnema Sylvestre Leaf Extract.

In addition or alternatively, the second, e.g., night-time, composition of the diet supplement may include Gymnema Sylvestre Leaf Extract. A serving of the second, e.g., nighttime, composition of the diet supplement may include from about 1 mg to about 2000 mg of Gymnema Sylvestre Leaf Extract. In a preferred dosage, a serving of the second, e.g., nighttime, composition of the diet supplement comprises about 133 mg of Gymnema Sylvestre Leaf Extract.

Enriched Soy Phospholipids

Soy phospholipids (also known as phosphatides) are a major class of lipids found in all living organisms. Phospholipids specific to living organisms include phosphatidylcholine, phosphatidylethanolamine, phosphatidylserine, and phosphatidylinositol. These phospholipids have been shown to aid in the absorption of fat and may improve cholesterol metabolism. A clinical trial demonstrated that administration of a preparation containing soy phospholipids to subjects lowered blood lipid-levels twice as much compared with a preparation devoid phospholipids (Hoie, L. H., et al., A double-blind placebo-controlled clinical trial compares the cholesterol-lowering effects of two different soy protein preparations in hypercholesterolemic subjects. Eur J Nutr, 2005. 44(2): p. 65-71.).

In addition, Phosphatidylserine has been demonstrated by way of clinical trials to reduce stress and lower cortisol levels (Monteleone, P., et al., Effects of phosphatidylserine on the neuroendocrine response to physical stress in humans. Neuroendocrinology, 1990. 52(3): p. 243-8.; Monteleone, P., et al., Blunting by chronic phosphatidylserine administration of the stress-induced activation of the hypothalamo-pituitary-adrenal axis in healthy men. Eur J Clin Pharmacol, 1992. 42(4): p. 385-8.) while positively affect mood (Benton, D., et al., The influence of phosphatidylserine supplementation on mood and heart rate when faced with an acute stressor. Nutr Neurosci, 2001. 4(3): p. 169-78.). Cortisol is a hormone, which among its many effects, acts to breakdown muscle. Phosphatidylserine also has been evidenced to increase exercise capacity (Kingsley, M. I., et al., Effects of phosphatidylserine on exercise capacity during cycling in active males. Med Sci Sports Exerc, 2006. 38(1): p. 64-71.).

Phosphatidylcholine is a source of choline, which is required for fat transport and normal brain functioning. It has been observed that choline levels drop as a result of athletic activity such as running (Conlay, L. A., L. A. Sabounjian, and R. J. Wurtman, Exercise and neuromodulators: choline and acetylcholine in marathon runners. Int J Sports Med, 1992. 13 Suppl 1: p. S141-2.). Therefore, choline supplementation may aid in athletic performance. When used in combination with carnitine and caffeine, choline can confer weight loss equal to exercise in animal models (Hongu, N. and D. S. Sachan, Caffeine, carnitine and choline supplementation of rats decreases body fat and serum leptin concentration as does exercise. J Nutr, 2000. 130(2): p. 152-7.). Clinical trials have demonstrated that choline combined with carnitine aids weight loss (Hongu, N. and D. S. Sachan, Carnitine and choline supplementation with exercise alter carnitine profiles, biochemical markers of fat metabolism and serum leptin concentration in healthy women. J Nutr, 2003.133(1): p. 84-9.).

The first, e.g., daytime, composition of the diet supplement may include enriched Soy Phospholipids. A serving of the first, e.g., daytime, composition of the diet supplement may include from about 0.1 mg to about 100 mg of enriched Soy Phospholipids. In a preferred dosage, a serving of the first, e.g., daytime, composition of the diet supplement comprises about 1 mg of enriched Soy Phospholipids.

Griffonia Simplicifolia

Griffonia Simplicifolia seed is a source of 5-hydroxy-L-tryptophan (5-HTP), which is a derivative of the amino acid tryptophan. In the body, tryptophan can be converted to 5-HTP which can, in turn, be converted into serotonin. Serotonin is a neurotransmitter which when low signals hunger, particularly carbohydrate cravings (Schweiger, U., et al., Macronutrient intake, plasma large neutral amino acids and mood during weight-reducing diets. J Neural Transm, 1986. 67(1-2): p. 77-86.).

Numerous clinical studies have found 5-HTP to be of benefit in the treatment of such conditions as depression, fibromyalgia, insomnia, headaches and obesity (reviewed in: Birdsall, T. C., 5-Hydroxytryptophan: a clinically-effective serotonin precursor. Altern Med Rev, 1998. 3(4): p. 271-80.). Furthermore, at least three clinical trials have demonstrated that 5-HTP can be used safely and effectively to aid in weight loss (Cangiano, C., et al., Eating behavior and adherence to dietary prescriptions in obese adult subjects treated with 5-hydroxytryptophan. Am J Clin Nutr, 1992. 56(5): p. 863-7.; Cangiano, C., et al., Effects of 5-hydroxytryptophan on eating behavior and adherence to dietary prescriptions in obese adult subjects. Adv Exp Med Biol, 1991. 294: p. 591-3.; Ceci, F., et al., The effects of oral 5-hydroxytryptophan administration on feeding behavior in obese adult female subjects. J Neural Transm, 1989. 76(2): p. 109-17.).

The first, e.g., daytime, composition of the diet supplement may include Griffonia Simplicifolia Seed Extract. A serving of the first, e.g., daytime, composition of the diet supplement may include from about 1 mg to about 1000 mg of Griffonia Simplicifolia Seed Extract. In a preferred dosage, a serving of the first, e.g., daytime, composition of the diet supplement comprises about 20 mg of Griffonia Simplicifolia Seed Extract.

Valerian Root

Valerian has traditionally been used to treat insomnia and stress. There may be a specific active compound, or alternatively a combination of compounds, contained in the Valerian Root Extract that is responsible for the beneficial effects of Valerian in aiding sleep. Compounds from Valerian Root are known to interact with the GABA, melatonin, and/or adenosine receptor systems through binding to certain melatonin and serotonin receptor subtypes (Abourashed, E. A., U. Koetter, and A. Brattstrom, In vitro binding experiments with a Valerian, hops and their fixed combination extract (Ze91019) to selected central nervous system receptors. Phytomedicine, 2004. 11(7-8): p. 633-8.), particularly the 5-HT_{5A} subtype (Dietz, B. M., et al., Valerian extract and valerenic acid are partial agonists of the 5-HT5a receptor in vitro. Brain Res Mol Brain Res, 2005.138(2): p. 191-7.).

Clinical trials have demonstrated that Valerian Root is safe and effective for treating stress and anxiety (Kohnen, R. and W. D. Oswald, The effects of valerian, propranolol, and their combination on activation, performance, and mood of healthy volunteers under social stress conditions. Pharmacopsychiatry, 1988. 21(6): p. 447-8.) as well as aiding sleep (Donath, F., et al., Critical evaluation of the effect of valerian extract on sleep structure and sleep quality. Pharmacopsychiatry, 2000. 33(2): p. 47-53.; Leathwood, P. D., et al., Aqueous extract of valerian root (Valeriana officinalis L.) improves sleep quality in man. Pharmacol Biochem Behav, 1982.17(1): p. 65-71.).

The second, e.g., night-time, composition of the diet supplement may include Valerian Root Extract (valeriana officianalis). A serving of the second, e.g., nighttime, composition of the diet supplement may include from about 1.0 mg to about 1000 mg of Valerian Root Extract (valeriana officianalis). In a preferred dosage, a serving of the second, e.g., nighttime, composition of the diet supplement comprises about 150 mg of Valerian Root Extract (valeriana officianalis).

Melatonin

Melatonin is a hormone produced by the pineal gland and is derived from tryptophan. It is involved in sleep regulation and is often used to treat sleep disorders such as insomnia and ‘jet lag’.

Melatonin has been shown to alleviate the symptoms of jet lag (Petrie, K., et al., Effect of melatonin on jet lag after long haul flights. Bmj, 1989. 298(6675): p. 705-7.; Suhner, A., et al., Effectiveness and tolerability of melatonin and zolpidem for the alleviation of jet lag. Aviat Space Environ Med, 2001. 72(7): p. 63846.) is able to shift circadian rhythms, aiding in sleep (Lewy, A. J., et al., Melatonin shifts human circadian rhythms according to a phase-response curve. Chronobiol Int, 1992. 9(5): p. 380-92.; Zhdanova, I. V., et al., Sleep-inducing effects of low doses of melatonin ingested in the evening. Clin Pharmacol Ther, 1995. 57(5): p. 552-8.; Hughes, R. J., R. L. Sack, and A. J. Lewy, The role of melatonin and circadian phase in age-related sleep-maintenance insomnia: assessment in a clinical trial of melatonin replacement. Sleep, 1998. 21(1): p. 52-68.). Melatonin has also be successfully been used to treat seasonal depression (Lewy, A. J., et al., Melatonin treatment of winter depression: a pilot study. Psychiatry Res, 1998. 77(1): p. 57-61.). Blood pressure and stress hormones have further been shown to be reduced by the daily oral administration of melatonin in healthy men (Arangino, S., et al., Effects of melatonin on vascular reactivity, catecholamine levels, and blood pressure in healthy men. Am J Cardiol, 1999. 83(9): p. 1417-9.). In mice, melatonin has been shown to reduce diet-induced weight gain (Prunet-Marcassus, B., et al., Melatonin reduces body weight gain in Sprague Dawley rats with diet-induced obesity. Endocrinology, 2003. 144(12): p. 5347-52.).

In addition or alternatively, the second, e.g., night-time, composition of the diet supplement may include Melatonin. A serving of the second, e.g., nighttime, composition of the diet supplement may include from about 0.1 mg to about 100 mg of Melatonin. In a preferred dosage, a serving of the second, e.g., nighttime, composition of the diet supplement comprises about 3 mg of Melatonin.

Chamomile

Chamomile is a popular herbal ingredient, wherein it is typically served as a tea. It is primarily known as a relaxant as well as a calming agent.

Ingesting chamomile tea results in indications of boosted immunity in humans (Wang, Y., et al., A metabonomic strategy for the detection of the metabolic effects of chamomile (Matricaria recutita L.) ingestion. J Agric Food Chem, 2005. 53(2): p. 191-6.). Studies in mice indicate that Chamomile has anti-allergic properties (Kobayashi, Y., et al., Dietary intake of the flower extracts of German chamomile (Matricaria recutita L.) inhibited compound 48/80-induced itch-scratch responses in mice. Phytomedicine, 2003. 10(8): p. 657-64.). Chamomile may also have anti-chemical dependence properties according to studies in rats (Gomaa, A., et al., Matricaria chamomilla extract inhibits both development of morphine dependence and expression of abstinence syndrome in rats. J Pharmacol Sci, 2003. 92(1): p. 50-5.).

Chamomile is known for inducing relaxation and possessing anxiolytic properties. Compounds present in Chamomile Extracts, namely Apigenin can bind to the benzodiazepine binding site in the gamma-aminobutyric acid receptor type A (GABA(A)) (Fernandez, S. P., et al., Synergistic interaction between hesperidin, a natural flavonoid, and diazepam. Eur. J. Pharmacol., 2005. 512(2-3): p. 189-98). The action of this ligand leads to sedation and a reduction in locomotor activity (Avallone, R. et al., Pharmacological profile of apigenin, a flavonoid isolated from Matricadia chamomilla. Biochem. Pharmacol. 2000. 59(11): p. 1387-94) without impairing memory or motor skills (Salgueiro, J. B., et al., Anxiolytic natural and synthetic flavonoid ligands of the central benzodiazepine receptor have no effect on memory tasks in rats. Pharmacol. Biochem. Behav., 1997. 58(4): p. 887-91).

The second, e.g., night-time, composition of the diet supplement may include chamomile, e.g., Chamomile from flower (matricaria recutita). A serving of the second, e.g., nighttime, composition of the diet supplement may include from about 0.1 mg to about 100 mg of Chamomile, e.g., Chamomile from flower (matricaria recutita). In a preferred dosage, a serving of the second, e.g., nighttime, composition of the diet supplement comprises about 1 mg of Chamomile, e.g., Chamomile from flower (matricaria recutita).

Passionflower

Passionflower is used as a sleep-aid in addition to a treatment for anxiety. Clinical studies have shown Passionflower has benefits as a treatment for opiate-withdrawal (Akhondzadeh, S., et al., Passionflower in the treatment of opiates withdrawal: a double-blind randomized controlled trial. J Clin Pharm Ther, 2001. 26(5): p. 369-73.) and anxiety treatment (Akhondzadeh, S., et al., Passionflower in the treatment of generalized anxiety: a pilot double-blind randomized controlled trial with oxazepam. J Clin Pharm Ther, 2001. 26(5): p. 363-7.). In rats, Passionflower has been shown to be a potent sedative (Capasso, A. and L. Sorrentino, Pharmacological studies on the sedative and hypnotic effect of Kava kava and Passiflora extracts combination. Phytomedicine, 2005. 12(1-2): p. 39-45.). Moreover, it contains a compound that is a known ligand for benzodiazepine receptors (Medina, J. H., et al., Chrysin (5,7-di-OH-flavone), a naturally-occurring ligand for benzodiazepine receptors, with anticonvulsant properties. Biochem Pharmacol, 1990. 40(10): p. 2227-31.), which typically convey relaxation signals.

The second, e.g., night-time, composition of the diet supplement may include passionflower, e.g., Passionflower from aerial parts (passiflora incarnate). A serving of the second, e.g., nighttime, composition of the diet supplement may include from about 0.01 g to about 1 g of passionflower, e.g., Passionflower from aerial parts (passiflora incarnate). In a preferred dosage, a serving of the second, e.g., nighttime, composition of the diet supplement comprises about 0.150 g of, e.g., Passionflower from aerial parts (passiflora incarnate).

The diet supplement according to this invention provides a method for causing fast weight loss, improving daytime energy, promoting nighttime relaxation and sleep, controlling appetite and/or increasing metabolism. Advantageously, consumption of the diet supplement is combined with a reduced calorie diet and a program of regular exercise.

According to various embodiments of the present invention, the diet supplement may be consumed in any form. For instance, the dosage form of the diet supplement may be provided as, e.g., a powder beverage mix, a liquid beverage, a ready-to-eat bar or drink product, a capsule, a tablet, a caplet, or as a dietary gel.

Preferably, the diet supplement is consumed by an individual in accordance with the following: As a dietary supplement, 2 caplets of the first, e.g., daytime, composition of the diet supplement may be taken with an 8 oz. glass of water approximately 30 to 60 minutes before breakfast. In addition, 2 caplets of the first, e.g., daytime, composition of the diet supplement may be taken with an 8 oz. glass of water approximately 30 to 60 minutes before lunch. Thereafter, 2 caplets of the second, e.g., nighttime, composition of the diet supplement may be taken with an 8 oz. glass of water approximately 30 to 60 minutes before dinner.

According to an embodiment of the present invention, the diet supplement is provided to a mother, e.g., a new mother shortly after childbirth, for causing fast weight loss, improving daytime energy, promoting nighttime relaxation and sleep, controlling appetite and/or increasing metabolism. For example, in an example embodiment, the first, e.g., daytime, composition of the diet supplement may be consumed by a new mother approximately 30 to 60 minutes before breakfast, wherein the first, e.g., daytime, composition of the diet supplement is formulated for causing fast weight loss, improving daytime energy, controlling appetite and/or increasing metabolism. In addition, the first, e.g., daytime, composition of the diet supplement may be consumed by a new mother approximately 30 to 60 minutes before lunch. Thereafter, the second, e.g., nighttime, composition of the diet supplement may be consumed by a new mother approximately 30 to 60 minutes before dinner, wherein the second, e.g., nighttime, composition of the diet supplement is formulated for causing fast weight loss, promoting nighttime relaxation and sleep, controlling appetite and/or increasing metabolism. In this manner, the various different compositions of the diet supplement may provide an appropriate benefit for all 24 hours of the day.

Furthermore, the dosage form of the diet supplement may be provided in accordance with customary processing techniques for herbal and/or dietary supplements in any of the forms mentioned above.

As set forth above, in one embodiment, the present invention provides, by the consumption of the diet supplement, a method for causing fast weight loss, improving daytime energy, promoting nighttime relaxation and sleep, controlling appetite and/or increasing metabolism. For example, the present invention provides, by the consumption of a first, e.g., daytime, composition of the diet supplement, a method for causing fast weight loss, improving daytime energy, controlling appetite and/or increasing metabolism. Furthermore, the present invention provides, by the consumption of a second, e.g., nighttime, composition of the diet supplement, a method for causing fast weight loss, promoting nighttime relaxation and sleep, controlling appetite and/or increasing metabolism.

The diet supplement set forth in the example embodiments herein may contain any appropriate number and type of excipients, as is well known in the art.

In addition, the present invention relates to a method of manufacturing a diet supplement for causing fast weight loss, improving daytime energy, promoting nighttime relaxation and sleep, controlling appetite and/or increasing metabolism. In accordance with one embodiment, the method of manufacturing the diet supplement may include the substeps of manufacturing a first, e.g., daytime, composition and a second, nighttime, composition of the diet supplement. For example, the substep of manufacturing a first, e.g., daytime, composition of the diet supplement may include the step of mixing one or more of Garcinia Cambogia, Green Tea Dry Leaf Extract (camellia sinensis), Anhydrous Caffeine, Chromium Polynicotinate, Gymnema Sylvestre Leaf Extract, Enriched Soy Phospholipids, and Griffonia Simplicifolia Seed Extract. The substep of manufacturing the first, e.g., daytime, composition of the diet supplement may also include the step of checking for uniformity/homogeneity. In addition, the substep of manufacturing the first, e.g., daytime, composition of the diet supplement, may include the step of aliquoting the mixture into a serving for compression into a caplet.

Furthermore, the substep of manufacturing a second, e.g., nighttime, composition of the diet supplement may include the step of mixing one or more of Garcinia Cambogia, Valerian Root Extract, Gymnema Sylvestre Leaf Extract, Melatonin, Chromium Polynicotinate, Chamomile and Passionflower. The substep of manufacturing the second, e.g., nighttime, composition of the diet supplement may also include the step of checking for uniformity/homogeneity. In addition, the substep of manufacturing the second, e.g., nighttime, composition of the diet supplement, may include the step of aliquoting the mixture into a serving for compression into a caplet.

Although the following example illustrates the practice of the present invention in one of its embodiments, the example should not be construed as limiting the scope of the invention. Other embodiments will be apparent to one skilled in the art from consideration of the specification of the following example.

### EXAMPLES

### Example 1

A diet supplement formulation for promoting fast weight loss and improving daytime energy is provided comprising the Calcium and Potassium double salt of Garcinia Cambogia Extract standardized to 60% Hydroxycitric Acid (1.55500 g), Extract Green Tea Leaf Extract (0.30444 g) standardized to 45% EGCG, 75% Catechins, 90% Polyphenols, Anhydrous Caffeine (0.07500 g), Chromium Polynicotinate (0.00133 g), Gymnema Sylvestre Extract (0.13300 g) standardized to 25% Gymnemic Acids, Enriched Soy Phospholipids (0.00100 g) standardized to 50% Phosphatidyl Serine, standardized to 4% Phosphatidyl Choline, 2% Phosphatidyl Ethanolamine and Griffonia Simplicifolia Extract (0.02000 g) standardized to 95% 5-HTP. The present embodiment, taken as a daytime supplement, may improve daytime energy, while controlling appetite and increasing metabolism.

Directions: As a diet supplement, 2 caplets of the daytime formulation of the diet supplement may be taken with an 8 oz. glass of water approximately 30 to 60 minutes before breakfast. As a second daily administration, 2 caplets of the daytime formulation of the diet supplement may be taken with an 8 oz. glass of water approximately 30 to 60 minutes before lunch.

### Example 2

A diet supplement for promoting fast weight loss and promoting nighttime relaxation is provided comprising the Calcium and Potassium double salt of Garcinia Cambogia Extract standardized to 60% Hydroxycitric Acid (1.182 g), Passionflower (0.150 g) supplying 3.5% flavanoids, Gymnema Sylvestre Extract (0.13300 g) standardized to 25% Gymnemic Acids, Melatonin (0.003 g), Chromium Polynicotinate (0.000133 g), Chamomile Extract (0.001 g) standardized to 1.2% Apigenin, Valerian Root Extract (0.0001 g) standardized to 0.8% Valerenic Acids in a formulation. The present embodiment, taken as a nighttime supplement, may induce relaxation and sleep, while controlling appetite and increasing metabolism.

Directions: As a diet supplement, 2 caplets of the nighttime formulation of the diet supplement may be taken with an 8 oz. glass of water approximately 30 to 60 minutes before dinner.

What is claimed is:

1. A diet supplement comprising at least the Calcium and Potassium double salt of Garcinia Cambogia Extract providing about 60% Hydroxycitric Acid, and one of Green Tea Leaf Extract and Melatonin.

2. The diet supplement of claim 1, further comprising Gymnema Sylvestre Extract and Chromium Polynicotinate.

3. The diet supplement of claim 2, further comprising Griffonia Simplicifolia Extract.

4. The diet supplement of claim 3, further comprising Enriched Soy Phospholipids.

5. The diet supplement of claim 4, further comprising Anhydrous Caffeine.

6. The diet supplement of claim 5, further comprising Passionflower.

7. The diet supplement of claim 6, further comprising Chamomile.

8. The diet supplement of claim 7, further comprising Valerian Root Extract.

9. A diet supplement comprising:
about 1.555 g of the Calcium and Potassium double salt of Garcinia Cambogia Extract providing about 60% Hydroxycitric Acid per serving;
about 0.30444 g of Green Tea Leaf Extract per serving;
about 0.133 g of Gymnema Sylvestre Extract per serving;
about 0.02 g of Griffonia Simplicifolia Extract per serving;
about 0.001 g of Enriched Soy Phospholipids per serving;
about 0.075 g of Anhydrous Caffeine per serving; and
about 0.00133 g of Chromium Polynicotinate.

10. A diet supplement comprising:
about 1.182 g of the Calcium and Potassium double salt of Garcinia Cambogia Extract providing about 60% Hydroxycitric Acid per serving;
about 0.15 g of Passionflower per serving;
about 0.133 g of Gymnema Sylvestre Extract per serving;
about 0.003 g of Melatonin per serving;
about 0.001 g of Chamomile Extract per serving;
about 0.0001 g of Valerian Root Extract per serving; and
about 0.00133 g Chromium Polynicotinate per serving.

11. A method for at least one of promoting fast weight loss, controlling appetite, improving daytime energy, promoting fast weight loss and increasing metabolism, comprising the step of administering to a human or animal the diet supplement of claim 1, wherein the diet supplement includes Green Tea Leaf Extract.

12. A method for increasing nighttime relaxation comprising the step of administering to a human or animal the diet supplement of claim 1, wherein the diet supplement includes Melatonin.

13. The method of claim 11, wherein the diet supplement is administered twice daily, once before breakfast and once before lunch.

14. The method of claim 12, wherein the diet supplement is administered once daily before dinner.

15. A method comprising the steps of:
administering to a human or animal twice daily, once before breakfast and once before lunch, the diet supplement of claim 1, wherein the diet supplement includes Green Tea Leaf Extract; and
administering once daily, before dinner, the diet supplement of claim 1, wherein the diet supplement includes Melatonin.

16. A kit comprising:
a first composition comprising at least the Calcium and Potassium double salt of Garcinia Cambogia Extract providing about 60% Hydroxycitric Acid, and Green Tea Leaf Extract; and
and a second composition comprising at least the Calcium and Potassium double salt of Garcinia Cambogia Extract providing about 60% Hydroxycitric Acid, and Melatonin.

17. A method comprising the step of administering the kit of claim 16 as a daily cycle of a diet supplement regime.

18. A method for promoting accelerated weight loss in new mothers comprising the step of administering the kit of claim 16.

19. A method for manufacturing a daytime formulation of a diet supplement comprising the step of:
forming a caplet comprising at least the Calcium Hydroxycitrate and Potassium Hydroxycitrate double salts of Garcinia Cambogia Extract providing at least 60% Hydroxycitric Acid, and Green Tea Extract;
mixing demineralized water, propylene glycol, titanium dioxide, riboflavin, hydroxypropyl methylcellulose, carboxymethylcellulose, maltodextrin, dextrose monohydrate, colorings and soy lecithin; and
using the mixture to sweet-coat the caplets.

20. A method for manufacturing a nighttime formulation of a diet supplement comprising the step of:
forming a caplet comprising at least the Calcium Hydroxycitrate and Potassium Hydroxycitrate double salts of Garcinia Cambogia Extract providing at least 60% Hydroxycitric Acid, and Melatonin;
mixing demineralized water, propylene glycol, titanium dioxide, riboflavin, hydroxypropyl methylcellulose, carboxymethylcellulose, maltodextrin, dextrose monohydrate, polysorbate 80, colorings and soy lecithin; and
using the mixture to sweet-coat the caplets.
